SEQUENCE LISTING (2 sequences)

SEQ ID NO: 1 - protein, 376 aa, Homo sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAAGAGPVVTPPEHLPMDSGGIDSSDSESDILLGILDNLDPVMFFKCPSPEPASLEELPEVYPEGPSSLPASLSLSVGTSSAKLEAINELIRFDHIYTKPLVLEIPSETESQANVVVKIEEAPLSPSENDHPEFIVSVKEEPVEDDLVPELGISNLLSSSHCPKPSSCLLDAYSDCGYGGSLSPFSDMSSLLGVNHSWEDTFANELFPQLISV

SEQ ID NO: 2 - protein, 261 aa, Homo sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAALRLRAPLQQVQAQLSPLQNISPWILAVLTLQIQSLISCWAFWTTWTQSCSSNALPQSLPAWRSSQRSTQKDPVPYQPPFLCQWGRHQPSWKPLMN

(19) United States
(12) Patent Application Publication
(10) Pub. No.: US 2022/0184175 A1
(43) Pub. Date: Jun. 16, 2022

(54) TREATMENT OF AGING OR AGE-RELATED DISORDERS USING XBP1

(71) Applicant: Universidad de Chile, Santiago (CL)

(72) Inventors: Claudio HETZ FLORES, Santiago (CL); Felipe CABRAL MIRANDA, Santiago (CL)

(21) Appl. No.: 17/426,613

(22) PCT Filed: Jan. 30, 2020
(86) PCT No.: PCT/IB2020/050754

§ 371 Date: Jul. 28, 2021

Related U.S. Application Data
(60) Provisional application No. 62/800,229, filed on Feb. 1, 2019.

(51) Int. Cl. A61K 38/17; A61K 35/76; A61K 47/64; A61P 25/28

(52) U.S. Cl. CPC A61K 38/1709; A61K 35/76; A61K 48/00; A61P 25/28; A61K 47/64

(57) ABSTRACT

Described is a targeted gene therapy for use in the delay or treatment of a symptomatic stage of aging and/or age-related disease in a subject, in particular to maintain or restore endoplasmic reticulum proteostasis. The gene therapy comprises the administration of a therapeutically effective amount of a pharmaceutically acceptable composition comprising X-box binding protein 1 (XBP1) or an agent that stimulates neuronal expression of XBP1 in the brain of the subject.

### TECHNICAL FIELD OF THE INVENTION

The present invention relates to the reduction in and reversal and treatment of symptoms of aging and age-related disorders using targeted gene therapy.

### BACKGROUND INFORMATION

The aging process is a stochastic and multifactorial phenomenon mediated both by environmental and genetic traits leading to organismal, tissue and cellular dysfunction and increased incidence of morbidity and death (1, 2, 3). The hallmarks of aging were recently defined as being cellular processes that mediate when and how we age. Geroscience is a generalized and progressive loss of function with the passage of time that makes us increasingly vulnerable to a broad suite of diseases: The Geroscience Hypothesis asserts that any intervention that retards the aging process will simultaneously delay the onset of multiple disease. Thus, the geroscience field looks at how the biology of aging influences the occurrence of disease.

Brain aging is the most relevant risk factor for the appearance of dementia and neurodegenerative diseases such as Alzheimer's disease (AD), Parkinson's disease (PD), fronto-temporal dementia (FTD) and amyotrophic lateral sclerosis (ALS) representing a major public health issue worldwide (3). In 2011, dementia was estimated to affect 35.6 million people around the world and the numbers are expected to reach about 135 million by 2050 (3, 5). Dementia is highlighted as an alarming economic issue for the future as it is recognized as being the largest factor driving the transition from independent living to dependence (4).

Several clinical trials are under development in the USA with the concept of attacking central hallmarks of aging, for example using senolytics drugs (Unity Biotechnology). The concept involves the idea that the health system will move from a “sick care” approach to a “health care” approach by intervening the biology of aging.

Although there is no consensuses in the literature regarding the molecular mechanisms that may explain the augmented disease rate in the elderly, a common feature of all these diseases is the accumulation of abnormal protein aggregates in the form of oligomers and inclusions, suggesting that cellular mechanisms controlling protein homeostasis (referred to as proteostasis) may underlay the etiology of these diseases and even drive age-associated cognitive dysfunction itself (5, 6). The proteostasis network controls the health of the proteome by integrating pathways involved in protein synthesis, folding, trafficking, secretion and degradation. Altered neuronal proteostasis is a molecular signature of the aged brain and plays a key role in the emergence of protein misfolding disorders. Aging drives a reduction in the buffering capacity of the proteostasis network thus increasing the probability of accumulating misfolded proteins in the brain. As almost one-third of the proteome is synthetized at the endoplasmic reticulum (ER), this organelle plays a key role in the maintenance of normal proteostasis. Indeed, the endoplasmic reticulum (ER) is a major component of the proteostasis network mediating the synthesis, folding and trafficking of most proteins in the brain (5, 6). Different harmful stimuli may trigger specific transcriptional programs and adaptive intracellular mechanisms following a condition known as endoplasmic reticulum stress (7). Accordantly, ER stress is a common feature of most neurodegenerative diseases which triggers ER stress sensor IRE1 and the activation of transcription factors. Such a response is collectively termed unfolded protein response (UPR) and one of its central mediators is the transcription factor XBP1s (X box binding protein) (7). This protein drives adaptive responses to increase the buffering capacity of the ER, maintaining cellular normal physiology and preventing function ablation (5, 6, 7). Data from simple model organisms including the nematode C. elegans and fly models indicate that altered proteostasis at the level of the ER is one of the major contributors to aging, impacting lifespan and organismal health-span (6). Since the presence of protein aggregates is observed in most neurodegenerative diseases, it is proposed that a decrease in the buffering capacity of the proteostasis network in the human brain may underlie the occurrence of pathological brain aging that will result in disease (7, 8). However, this hypothesis has not been tested yet in mammals and the contribution of the UPR to brain aging remains unexplored.

The functional contribution of the UPR to protein misfolding disorders affecting the nervous system has recently been defined and possible new avenues of therapeutic interventions suggested (9, 10, 11, 12), In this context, XBP1s has been shown to be a significant mediator of brain disease emergence in different age-associated diseases. In support of this, genomic screens have identified a polymorphism in the XBP1 promoter as a risk factor for Alzheimer's disease (13), bipolar disorder and schizophrenia in certain populations (7, 13). Additionally, a functional contribution of XBP1s in the normal physiology of the brain has been identified as knockout animals for this protein present decreased cognitive capacity at behavioral and molecular level and increased expression of XBP1s mediated by viral vectors in the brain was sufficient to ameliorate cognition (14). In line with these findings, other groups have shown that Xbp1 mRNA is upregulated in animals exposed to enriched environments (15) and is activated locally in neurites in response to brain-derived neurotrophic factor (BDNF) treatment, enhancing neurite outgrowth in vitro (16, 17).

The dysfunction of the UPR triggering has recently emerged as prominent feature of the aging process in different animal models. For example, exposure of aged C. elegans to ER stress-inducing agents indicated that the ability of cells to activate the UPR is significantly ablated when compared to young animals, suggesting that aging disrupts the ER stress response (6, 18). Different studies performed in both vertebrate and invertebrate models provided correlations indicating that distinct components of the UPR are altered, or exhibit altered profile, at transcription and/or translational levels in different tissues and cell types of aged organisms (6). Loss-of-function studies in C. elegans demonstrated that lifespan extension conferred by XBP1 expression is dependent on insulin/IGF-1-FOXO signaling, a classical pathway associated with aging (18). Remarkably, the selective overexpression of XBP1s in neurons of C. elegans was able to increase lifespan up to 30%, representing one of the strongest aging genetic modulators described so far for this specie (18). Despite these discoveries, no data is available about the possible role of ER proteostasis to mammalian brain aging at the functional and molecular level.

Accordingly, the present invention seeks to prevent, reduce, reverse or delay natural decay in the basal cognitive and motor capacity of aged animal using a gene therapy approach to target and improve ER proteostasis.

### SUMMARY OF THE INVENTION

In one aspect the present invention resides in a method for the delay or treatment of a symptomatic stage of aging in a subject, the method comprising administering to the subject a therapeutically effective amount of a pharmaceutically acceptable composition comprising X-box binding protein 1 (XBP1) or an agent that stimulates or induces neuronal expression or over-expression of XBP1s in the brain, preferably the hippocampus, to maintain or restore endoplasmic reticulum proteostasis in the subject.

In another aspect, the present invention resides in a method for treatment of an age-related disorder in a subject, the method comprising administering to the subject a therapeutically effective amount of (preferably a pharmaceutically acceptable composition comprising) X-box binding protein 1 (XBP1) or an agent that stimulates or induces (preferably neuronal) expression or over-expression of XBP1s in the brain, preferably the hippocampus, preferably to maintain or restore endoplasmic reticulum proteostasis in the subject.

In another aspect, the present invention resides in a pharmaceutically acceptable composition comprising X-box binding protein 1 (XBP1s) or an agent that stimulates neuronal expression of XBP1s and a pharmaceutically acceptable carrier for use in the maintenance or restoration of endoplasmic reticulum proteostasis in the brain, preferably the hippocampus, of a subject. Accordingly, the pharmaceutically acceptable composition described herein is used for the delay or treatment of a symptomatic stage of aging.

In a further aspect, the present invention resides in the use of a pharmaceutically acceptable composition comprising X-box binding protein 1 (XBP1s) or an agent that stimulates neuronal expression of XBP1s and a pharmaceutically acceptable carrier in the manufacture of a medicament for the maintenance or restoration of endoplasmic reticulum proteostasis in the brain, preferably the hippocampus, of a subject. The maintenance or restoration of endoplasmic reticulum proteostasis in the brain manifests as the delay or treatment of a symptomatic stage of aging.

The aim of the present invention is to provide a therapeutic substantially to prevent, reduce, reverse or delay the natural decay in brain function that is typically seen with age and often translates into the development of diseases including dementia,

Alzheimer's and Parkinson's. The symptomatic stage of aging typically manifests itself as a decline in basal motor and cognitive function and capacity which, in one hypothesis, is believed to be caused by altered neuronal proteostasis, particularly in the endoplasmic reticulum (ER). Therefore, without wishing to be bound by theory, maintenance or restoration of normal proteostasis in the aged brain is expected to enhance the health and life quality in an elderly population, thereby delaying or reducing the likelihood of the onset of age-related diseases. The terms “aging”, “aged” and “elderly” refer to subjects who have reached the middle stage of life and beyond and also encompasses subjects who present age-related symptoms, such as a decline in basal motor and cognitive function and capacity, at any stage of life. In some embodiments, the subject is a human subject aged 40 years or more, 50 years or more, 60 years or more, 70 years or more, 40 to 100 years, 50 to 90 years or 60 to 80 years.

In some embodiments, the present invention may be applied to prevent or treat an age-related disorder or disease, e.g. an age-related disorder of the brain or an age-related neurological disorder. Thus, in one embodiment, the method is used to treat or prevent age-related cognitive decline. In another embodiment, the method is used to treat accelerated aging, i.e. progeria.

By “pharmaceutically acceptable” is meant a material that is not biologically or otherwise undesirable, e.g., the material may be administered to a subject without causing any undesirable biological effects. Thus, such a pharmaceutical composition may be used, for example, in transfection of a cell ex vivo or in administering a composition, viral particle or cell directly to a subject.

A “therapeutically-effective” amount as used herein is an amount that is sufficient to alleviate (e.g., prevent, mitigate, decrease, reduce, reverse) at least one of the symptoms associated with a disease state. Alternatively stated, a “therapeutically-effective” amount is an amount that is sufficient to provide some improvement in the condition of the subject. A “therapeutically effective amount” will fall in a relatively broad range that can be determined through experimentation and/or clinical trials. In one example, for instance in the case of an adeno-associated virus vector that induces expression of XBP1 in neuronal tissue following in vivo injection, a therapeutically effective dose may be in the order of from about 10⁵ to about 10⁴⁰ viral genomes/kg of weight, e.g., from about 10⁹ to 10³⁰ viral genomes/kg. For in vitro transduction, an effective amount of AAV virions to be delivered to cells may be in the order of from about 10⁸ to about 10¹⁵ copies of viral genome per ml. Other effective dosages may be readily established by one of ordinary skill in the art through routine trials establishing dose response curves.

In some embodiments, more than one administration (e.g., two, three, four or more administrations) may be employed to achieve the desired level of therapeutic effect over a period of various intervals, e.g., daily, weekly, monthly, yearly, etc.

The present disclosure provides a pharmaceutical composition comprising: a) an active ingredient as described herein; and b) a pharmaceutically acceptable carrier, diluent, excipient, or buffer. In some embodiments, the pharmaceutically acceptable carrier, diluent, excipient, or buffer is suitable for use in a human. Such excipients, carriers, diluents, and buffers include any pharmaceutical agent that can be administered without undue toxicity. Pharmaceutically acceptable excipients include, but are not limited to, liquids such as water, saline, glycerol and ethanol. For injection, the carrier will typically be a liquid. For other methods of administration, the carrier may be either solid or liquid, such as sterile, pyrogen-free water or sterile pyrogen-free phosphate-buffered saline solution. For inhalation administration, the carrier will be respirable, and will preferably be in solid or liquid particulate form. As an injection medium, it is preferred to use water that contains the additives usual for injection solutions, such as stabilizing agents, salts or saline, and/or buffers.

The pharmaceutically acceptable composition may include salts, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7(th) ed.,

Lippincott, Williams, & Wilkins; and Handbook of Pharmaceutical Excipients (2000) A. H. Kibbe et al., eds., 3 rd ed. Amer. Pharmaceutical Assoc.

In one embodiment, the pharmaceutically acceptable composition comprises X-box binding protein 1 (XBP1) or a mimic thereof. In another embodiment, the pharmaceutically acceptable composition comprises an agent that stimulates expression (preferably neuronal expression) of a polypeptide comprising XBP1. The term XBP1 as used herein includes functional fragments, variants and homologues thereof, including e.g. splice variants XBP1s and XBP1u, e.g. as described in WO2016/106458. The amino acid sequence of human XBP1s is disclosed in, for example, NCBI database accession no. NP_001073007.1 and is shown in SEQ ID NO:1. The amino acid sequence of human XBP1u is disclosed in, for example, NCBI database accession no. NP_005071.2 and is shown in SEQ ID NO:2. Thus in some embodiments, the term “XBP1” includes a polypeptide having e.g. at least 85%, at least 90%, at least 99% or at least 99% sequence identity to SEQ ID NO:1 or SEQ ID NO:2. Typically the polypeptide has this degree of sequence identity over at least 50, at least 100, at least 200 or at least 300 amino acid residues, or over the full length of the polypeptide. Functional fragments of XBP1 (e.g. XBP1s or XBP1u) typically comprise at least 50, at least 100, at least 200 or at least 300 amino acid residues of SEQ ID NO:1 or SEQ ID NO:2.

The terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. A polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters:

Mismatch Penalty: 1.00;

Gap Penalty: 1.00;

Gap Size Penalty: 0.33; and

Joining Penalty: 30.0.

In some embodiments, the induced polypeptide may comprise XBP1 fused to a further polypeptide sequence. For instance, the agent may induce expression of a UPRplus fusion protein as described in WO2017/075729. UPR refers to the universal protein response and transcription factors involved therein. Thus, in one embodiment the agent may induce expression of a fusion protein comprising XBP1 and an additional UPR transcription factor (e.g. ATF6), optionally joined by a linker.

Preferably the agent stimulates neuronal expression of a polypeptide comprising XBP1s.

In preferred embodiments, the agent is an adeno-associated virus vector (AAV vector). The term AAV covers all subtypes and both naturally occurring and recombinant forms, except where required otherwise. The term AAV includes, for example, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10, including AAVrh10), AAV type 12 (AAV-12), avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, and so on.

The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC-002077 (AAV-1), AF063497 (AAV-1), NC-001401 (AAV-2), AF043303 (AAV-2), NC-001729 (AAV-3), NC-001829 (AAV-4), U89790 (AAV-4), NC-006152 (AAV-5), AF513851 (AAV-7), AF513852 (AAV-8), and NC-006261 (AAV-8); the disclosures of which are incorporated by reference herein. See also, e.g., Srivistava et al. (1983) J. Virology 45:555; Chiorini et al. (1997) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al.,(1986) J. Virol. 58:921; Gao et al. (2002) Proc. Nat. Acad. Sci. USA 99: 11854; Mori et al. (2004) Virology 33:375-383; international patent publications WO 00/28061, WO 99/61601, WO 98/11244; and U.S. Pat. No. 6,156,303. In one example, the sequence of the genome of an AAV2 serotype is as defined in GenBank access number AF043303.1.

The AAV vector as used herein may be a recombinant AAV vector comprising a polynucleotide sequence not of AAV origin (i.e., a polynucleotide heterologous to AAV), typically a sequence of interest for the genetic transformation of a cell, in particular a sequence encoding XBP1 (e.g. XBP1s or a polypeptide comprising

XBP1s). In some embodiments, the heterologous polynucleotide may be flanked by at least one, and sometimes by two, AAV inverted terminal repeat sequences (ITRs).

“Recombinant,” as used herein means that the vector, polynucleotide, polypeptide or cell is the product of various combinations of cloning, restriction or ligation steps (e.g. relating to a polynucleotide or polypeptide comprised therein), and/or other procedures that result in a construct that is distinct from a product found in nature. A recombinant virus or vector is a viral particle comprising a recombinant polynucleotide. The terms respectively include replicates of the original polynucleotide construct and progeny of the original virus construct.

In preferred embodiments, the AAV vector induces expression of a polypeptide comprising XBP1 (e.g. XBP1s) in the central nervous system, preferably in neurons. The AAV vector may, for example, induce neuronal expression of XBP1. In some embodiments, the AAV vector may, for example be of the serotype AAV2, AAV6, AAV7, AAV8 or AAV9, preferably AAV2 or AAV6.

In some embodiments, a nucleotide sequence encoding a polypeptide comprising XBP1 is operably linked to a constitutive promoter. In other embodiments, a nucleotide sequence encoding a polypeptide comprising XBP1 is operably linked to an inducible promoter. In some instances, a nucleotide sequence encoding a polypeptide comprising XBP1 is operably linked to a tissue specific or cell type specific regulatory element.

For example, in some instances, a nucleotide sequence encoding a polypeptide comprising XBP1 is operably linked to a neuron-specific regulatory element e.g., a regulatory element that confers selective expression of the operably linked gene in a neuron. Suitable neuronal-specific promoters include e.g. neuron-specific enolase (NSE) promoter, Andersen et al. Cell. Mol. Neurobiol., 13:503-15 (1993; neurofilament light-chain gene promoter, Piccioli et al., Proc. Natl. Acad. Sci. USA, 88:5611-5 (1991); and the neuron-specific vgf gene promoter, Piccioli et al., Neuron, 15:373-84 (1995)]; among others.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

The term “promoters” or “promoter” as used herein can refer to a DNA sequence that is located adjacent to a DNA sequence that encodes a recombinant product. A promoter is preferably linked operatively to an adjacent DNA sequence. A promoter typically increases an amount of recombinant product expressed from a DNA sequence as compared to an amount of the expressed recombinant product when no promoter exists.

The term “enhancers” or “enhancer” as used herein can refer to a DNA sequence that is located adjacent to the DNA sequence that encodes a recombinant product. Enhancer elements are typically located upstream of a promoter element or can be located downstream of or within a coding DNA sequence (e.g., a DNA sequence transcribed or translated into a recombinant product or products). Hence, an enhancer element can be located 100 base pairs, 200 base pairs, or 300 or more base pairs upstream or downstream of a DNA sequence that encodes recombinant product. Enhancer elements can increase an amount of recombinant product expressed from a DNA sequence above increased expression afforded by a promoter element. Multiple enhancer elements are readily available to persons of ordinary skill in the art.

One example of a suitable agent is an adeno-associated virus (e.g. an AAV6) vector that encodes neuronal expression of a polypeptide comprising XBP1s, e.g. as described in WO2016/106458, the content of which is incorporated herein in its entirety. In another embodiment the agent is an AAV2 vector that encodes neuronal expression of XBP1 in the CNS or an AAV vector that induces expression of XBP1 in motor neurons, e.g. an AAV vector encoding XBP1s as described in WO2017/059554, the content of which is incorporated herein in its entirety. In another embodiment the agent is an AAV vector that induces neuronal expression of a fusion protein comprising UPRplus, e.g. the AAV vector induces neuronal expression of a fusion protein comprising XBP1 and ATF6 joined by a linker, e.g. as described in WO2017/075729, the content of which is incorporated herein in its entirety. These AAVs and other viral vectors are particularly suitable as they offer “vaccination-like” treatment strategies to reach the nervous system with high efficacy and safety without the need of intracerebral injections. It will be appreciated that the invention encompasses other agents that stimulate neuronal expression of XBP1s, such as synthetic and biological agonists/ligands and factors that target the proteome and/or the genome.

In one aspect the present invention encompasses the delivery of a gene product to a tissue or cell (e.g. a neuronal tissue or cell) in a subject. The gene product may be a polypeptide or an interfering RNA (e.g., an shRNA, an siRNA, and the like), or an aptamer. In one embodiment, the cell may be, for example, be a stem cell, neural cell, glial cell (e.g. astrocytes, oligodendrocytes and so on) or dendritic cell. Similarly, the tissue may, for example, be central or peripheral nervous system tissue (e.g. brain or neuronal tissue).

The present invention finds use in both veterinary and medical applications. Preferred subjects are mammals, with the term as used herein including, but not limited to, humans, bovines, ovines, caprines, equines, felines, canines, lagomorphs, cavies, rodents, etc. Human subjects are the most preferred.

The term “administered locally” as used herein means that the pharmaceutical compositions of the invention are administered to the subject in or close to a specific site.

The term “administered systemically” and “systemic administration” as used herein means that the pharmaceutical compositions of this invention are administered to a subject in a non-localized manner. The system administration of pharmaceutical compositions of the invention may reach various organs or tissues of the subject's entire body or may reach specific organs or tissues of the subject. For example, the intravenous administration of a pharmaceutical composition of the invention may give rise to transduction in more than one tissue or organ in a subject. In one embodiment, systemic administration is via means that enables the composition of the invention to cross the blood brain barrier.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein encompasses any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease.

The present disclosure provides a method of treating a disease (e.g. a neurological disease), the method comprising administering to an individual in need thereof an effective amount of a pharmaceutically acceptable composition as described above. The composition may be administered via intracranial injection, intracerebral injection, intraventricular injection, intrathecal injection, intravenous injection or by any other convenient mode or route of administration.

Further exemplary modes of administration include oral, rectal, transmucosal, topical, transdermal, inhalation, parenteral (e.g., intravenous, subcutaneous, intradermal, intramuscular, and intraarticular) administration, and the like, as well as direct tissue or organ injection, alternatively, intrathecal, direct intramuscular, intraventricular, intravenous, intraperitoneal, intranasal, or intraocular injections. Injectables can be prepared in conventional forms, either as liquid solutions or suspensions, solid forms suitable for solution or suspensions in liquid prior to injection, or as emulsions. Alternatively, one may administer the virus in a local rather than systemic manner, for example in a depot or sustained-release formation.

Where used, recombinant virus vectors are preferably administered to the subject in an amount that is sufficient to result in infection (or transduction) and expression of the heterologous nucleic acid sequence in cells (e.g. neuronal cells) of the subject. Preferably the target cells are neural cells, including cells of the central and peripheral nervous systems, in particular, brain cells.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference in their entirety to disclose and describe the methods and/or materials in connection with which the publications are cited.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The following examples are provided to illustrate certain embodiments of the invention and they are not intended to limit the invention in any way.

### BRIEF DESCRIPTION OF THE DRAWINGS

FIG. 1 demonstrates that AAV2-XBP1s gene delivery to aged mouse hippocampus reverts cognitive, functional and morphological changes associated to normal aging.

FIG. 1A) Representative photomicrography of an aged wild type mice injected with AA2-XBP1s in CA1 region of the hippocampus (magnification 40×). FIG. 1B) Novel Object Location test, 1C) Novel Object Recognition test, and 1D) Barnes Maze test, indicate that aged mice injected with AAV2-XBP1s in the hippocampus (n=15) present normal cognition when compared to aged control animals (n=14). (p=0.0130; p=0.0218; p=0.065 in Day 2 of Barnes Maze).

FIG. 2 demonstrates that AAV2-XBP1s gene delivery to aged mouse hippocampus reverts cognitive, functional and morphological changes associated to normal aging.

FIGS. 2A, 2B, 2C, and 2D) Dendritic spikes quantification shows increased proportion of total and mushroom-like dendritic spikes in the CA1 region of aged mice injected with AAV-XBP1s when compared to controls (n=5, 6; n=8 dendrites per animal; p=0.0092).

FIG. 3 demonstrates that AAV2-XBP1s gene delivery to aged mouse hippocampus reverts cognitive, functional and morphological changes associated to normal aging.

FIGS. 3A and 3B) Field excitatory post-synaptic potentials increase following stimuli for 60 minutes in slices from aged mice injected with AAV-XBP1s (n=9, 28) or AAV-mock (n=6, 17) slices (p=0.0245, Unpaired t-test followed by Dunnett's post test).

FIG. 4 demonstrates that AAV2-XBP1s gene delivery to aged mouse hippocampus reverts cognitive, functional and morphological changes associated to normal aging.

FIGS. 4A, 4B, 4C, 4D, and 4E) Firing rate of spontaneous hippocam pal activity (SA) in slices from aged AAV-XBP1s or AAV-mock animals; (n=9, 6; p<0.0001) treated with picrotoxin (PTX) or untreated (SA). Frequency distribution in function of time (down) of firing rate and spikes bursts (%).

FIG. 5 shows that IRE1 ablation in the brain accelerates and exacerbates age-associated cognitive and motor decline in mammals without influencing behavior during youth. FIG. 5A) New Object Recognition test indicates that aged wild type (n=17) mice fail to recognize new presented objects when compared to middle aged (n=24) or young animals (n=14; p<0.0001, One-way ANOVA followed by Tukey's post-test). IRE1^{cKO} mice, on the other hand, when evaluated by the same test present decreased capacity to recognize novel objects during middle age but not during youth (n=23, 16; two-way ANOVA followed by Sidak's post test, p=0.0004).

FIG. 5B) The Barnes Maze test indicates that on test day 5, aged IRE1^{cKO} mice make more mistakes until they can find the target hole when compared to aged wild types, which does not happen during middle age or youth (n=9, 9, 9, 9, 10, 8; Unpaired t Student's test, p=0.0092). FIG. 5C) Contextual fear conditioning test indicates that young IRE1^{cKO} mice present normal freezing responses compared to wild types (n=7, 10; unpaired t Student's Test, p=0.2305). Middle aged knock out, however, present decreased freezing responses compared to middle aged wild type (n=9, 14; unpaired t Student's test, p=0.0012). FIG. 5D) Hang test was performed in wild type and IRE1^{cKO} mice at different ages to evaluate motor performance. Old IRE1^{cKO} mice (n=18) were compared with aged WT animals (n=15) and presented decreased motor function at this age (unpaired t Student's test, p=0.0002) which was not observed during middle age (n=11, 15; p=0.6121) or youth (n=10, 12; p=0.6434). FIG. 5E) The rotarod test was used to evaluate motor performance in wild type (n=16, 8, 18) and IRE1cKO mice (n=12, 8, 6) during youth, middle age or advanced age. Latencies to fall were plotted and compared within each age and showed that only aged animals presented decreased function following IRE1 ablation in the brain (n=17, 8; unpaired Student's t test, p=0.0351). FIG. 5F) Young IRE1^{cKO} mice present normal behavior as shown by the New Object Location test (n=17, 22; p=0.1741). During middle age, animals lose the capacity to discriminate between objects (n=22, 13; p=0.104).

FIG. 6 shows that IRE1 ablation in the brain accelerates and exacerbates age-associated cognitive and motor decline in mammals without influencing behavior during youth. FIGS. 6A, 6B, and 6C) Western Blots for PSD95, synaptophysin and SNAP-25 indicates a decreased Synaptophysin and SNAP-25 content in the hippocampus and cerebral cortex of middle aged and aged IRE1 Nest KO mice when compared to controls (n=5, 6, 6, 6, p values indicated).

FIG. 7 shows that IRE1 ablation in the brain accelerates and exacerbates age-associated cognitive and motor decline in mammals without influencing behavior during youth. FIGS. 7A, 7B, and 7C) Compound Muscle Action Potentials (CMAPs) indicate there is no difference in voltage amplitudes comparing IRE1^{cKO} mice with wild types during the course of aging (n=11, 11, 10, 10, 11, 7; p>0.05).

FIG. 8 shows that IRE1 ablation in the brain accelerates and exacerbates age-associated cognitive and motor decline in mammals without influencing behavior during youth. FIGS. 8A and 8B) Neuromuscular junction evaluation indicate there is no difference in morphology when comparing IRE1^{cKO} with wild type mice (n=4, 4).

FIG. 9 shows that XBP1s overexpression in the brain using transgenic mice prevents the emergence of age-associated motor and cognitive dysfunction in mammals. FIG. 9A) New Object Location test indicates that middle aged (n=12, 13; p=0.0007) and aged (n=15, 9; p=0.0417) XBP1s transgenic mice can discriminate between new located objects when compared to age-matched animals. FIG. 9B) New Object Recognition test indicates that aged XBP1s transgenic mice present normal behavior as expected for young wild type mice (n=17, 12; p=0.0130). FIG. 9C) Barnes Maze test shows that aged XBP1s transgenic mice (n=8) spend less time to get to the target hole when compared to aged wild type mice (n=10) in day 1 (p=0.0039), day 2 (p=0.0028), day 3(p=0.018) and day 4 (p=0.003). FIG. 9D) Middle aged (n=8, 8; p=0.040) and aged (n=8, 8; p=0.0056) XBP1s transgenic mice exhibit better scores in the hang test when compared to age-matched wild types. No significant difference is observed during youth (n=8, 8; p=0.107). FIG. 9E) Rotarod test indicates that aged XBP1s transgenic mice (n=10) show increased latency to fall from the rod when compared to aged wild types (n=17) as there is no significant difference during youth (n=16, 12; p=0.566). FIG. 9F) No significant difference was encountered in the morphology of aged XBP1s transgenic mice when compared to wild types (n=7, 7).

FIG. 10 shows that XBP1s overexpression in the brain using transgenic mice prevents the emergence of age-associated motor and cognitive dysfunction in mammals. FIGS. 10A, 10B, and 10C) CMAP readings indicate there is a decreased amplitude in the muscles of young XBP1s transgenic mice (n=6) when compared to young wild types mice (n=10) that is not maintained during aging when evaluating middle aged (n=11, 8) or aged (n=10, 8) mice.

### EXAMPLES

As the aging process is the most important risk factor to the emergence of dementia and neurodegenerative diseases, a technology has been developed and evaluated to revert the decay in the buffering capacity of the brain during aging, aiming to revert the normal decay in brain function that may even translate into the development of dementia or Alzheimer. In this scenario, a viral vector (adeno-associated virus) has been developed to drive the ectopic overexpression of an active from of the transcription factor XBP1 in mammals. XBP1 is a master regulator of the unfolded protein response (UPR) that establishes pro-survival and repair gene expression programs to restore proteostasis, in addition to controlling synaptic function by directly controlling the expression of synaptic proteins and neurotrophins such as brain-derived neurotrophic factor (BDNF).

### Example 1: Delivery of AAV2-XBP1s into the Hippocampus of Aged Mice Reverts Cognitive Decline

In this example, proteostasis was restored to the brain of aged mice. A local gain-of-function approach was used to assess whether the direct delivery of XBP1s using AAV-based gene therapy to the brain could block the dysfunction in cognition observed during the aging process.

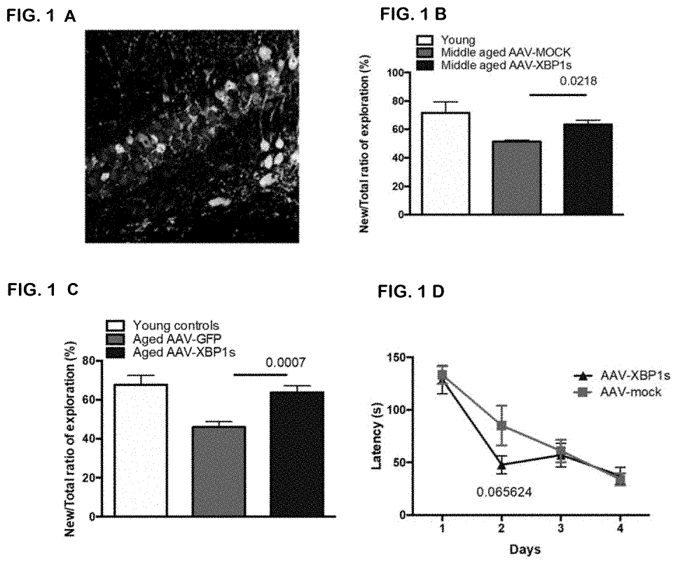

Middle aged and aged wild type animals were stereotaxically injected with AAV2-XBP1s (prepared e.g. as described in WO 2017/059554) in both hippocampus (FIG. 1A), as this brain region is directly associated to the process of learning and memory, crucial to a normal phenotype in all cognitive tests implemented here. Extraordinarily, middle aged and old mice overexpressing XBP1s presented cognitive function comparable to young animals, which is not observed in aged AAV-mock injected mice (FIGS. 1B, 1C, and 1D). This finding indicates that gene delivery of AAV2-XBP1s to the brain is sufficient to revert age-associated cognitive dysfunction in wild type animals.

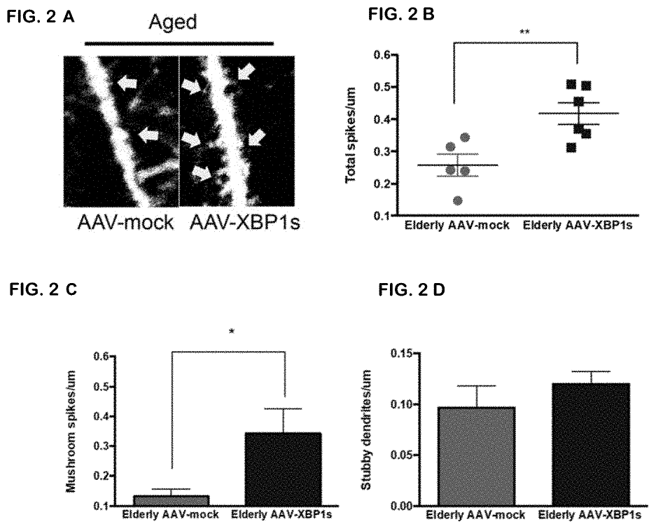

The distribution of mushroom like dendritic spikes was then analyzed, as its counting is associated with increased cognitive capacity in brain circuits and represents a mechanism of synaptic plasticity. The results indicate an increased number of dendritic spikes/rea in mice that received AAV2-XBP1s treatment in the hippocampus (FIGS. 2A to 2D).

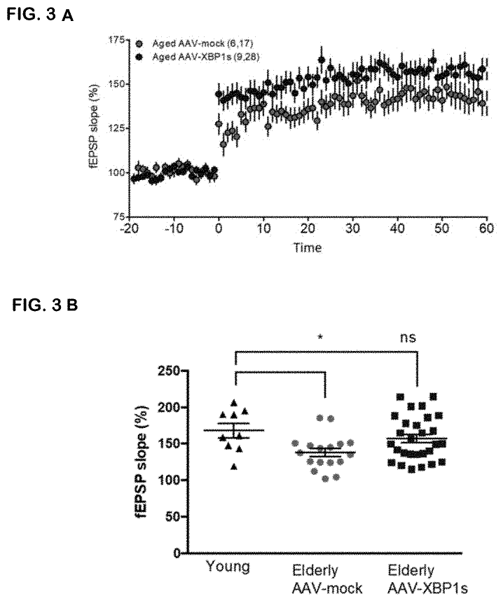

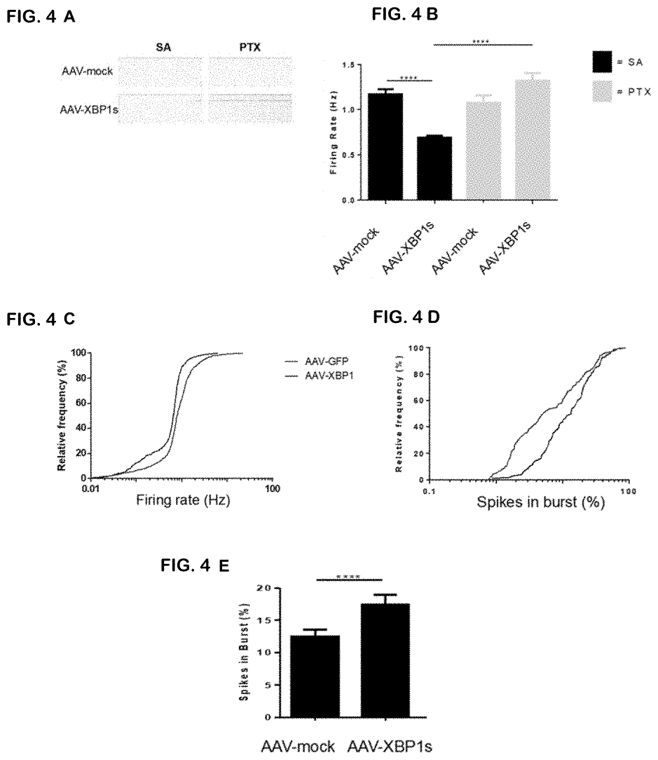

Finally, electrophysiological properties of hippocampal circuitry were evaluated in aged animals treated with AAV2-XBP1s. After electrical stimulation of Schaffer Collateral Fibers, an increased long-term potentiation (LTP) was observed in postsynaptic terminals which is sustained one hour after stimuli (FIGS. 3A and 3B). Interestingly, the slope increase is higher in AAV2-XBP1s injected animals when compared to aged AAV2-mock injected. When evaluating the basal firing rate in hippocampal slices, a decreased response was observed in AAV2-XBP1s injected animals when compared to controls, suggesting that inhibitory circuits are preserved in aged mice following XBP1s overexpression (FIGS. 4A to 4E).

AAV2-XBP1s was delivered into the hippocampus of aged mice that already presented a cognitive decline. The results indicated the aged animals showed a full reversion of the age-associated cognitive decline when compared to controls animals, recovering synaptic capacity. Thus, this data indicates that XBP1s gene delivery to the brain mediated by AAV infection reverts age-associated dysfunction of the brain at behavioral, morphological and functional levels.

### Example 2: Bursting UPR Proteostasis by Expressing XBP1s Improved the Quality of Brain Aging at the Level of Cognitive Function

In this example, the function of the IRE1-XBP1s axis in the aging process of the mammalian brain was evaluated at the motor and cognitive level using different behavioral approaches. The results show that genetically disrupting ER stress sensor IRE1 function in the brain accelerates and exacerbates age-associated phenotypes. Importantly, increasing XBP1s content in the brain, specifically in the hippocampus, is sufficient to slow down or even block cognitive and motor dysfunction in elderly mice. These results correlate with altered electrophysiological responses, synaptic proteins content and brain-derived neuronal factor (BDNF) levels in the brain of aged mice.

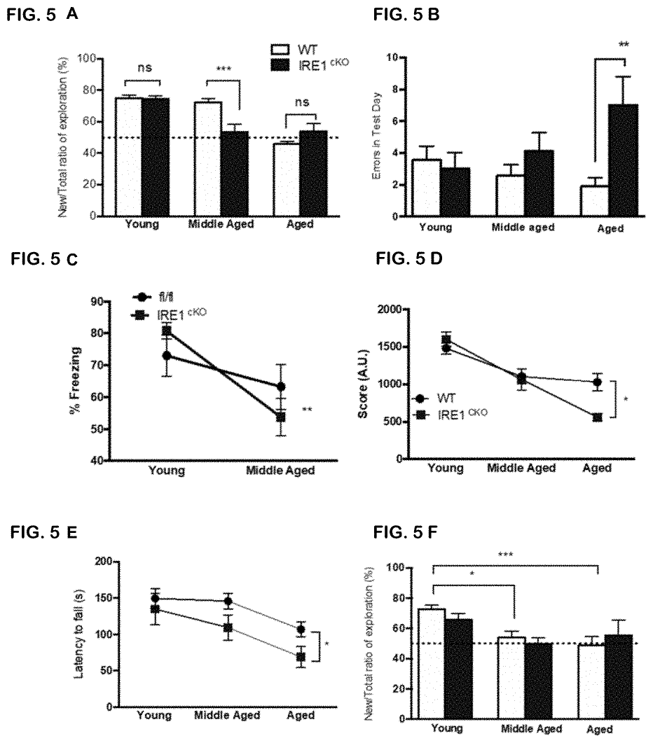

To evaluate if genetic disruption of the IRE1-XBP1s pathway in the brain would influence age associated cognitive and motor dysfunction, conditional transgenic mice with a mutated form of IRE1 lacking the RNase domain (IRE1^{cKO}) in the brain were used as previously reported (12). These animals present no mRNA splicing of XBP1s in brain tissue indicating that the pathway was fully inactivated. When comparing wild types with IRE1^{cKO} mice during youth, no differences were detected in any of the behavioral tests implemented (FIGS. 5A to 5F, 6A to 6C, 7A to 7C, 8A and 8B.). Age matched wild type animals were then compared with different conditional transgenic mice with gain or loss-of-function experiments to evaluate whether genetic manipulation of IRE1-XBP1s axis in the brain may alter age-associated cognitive disruption.

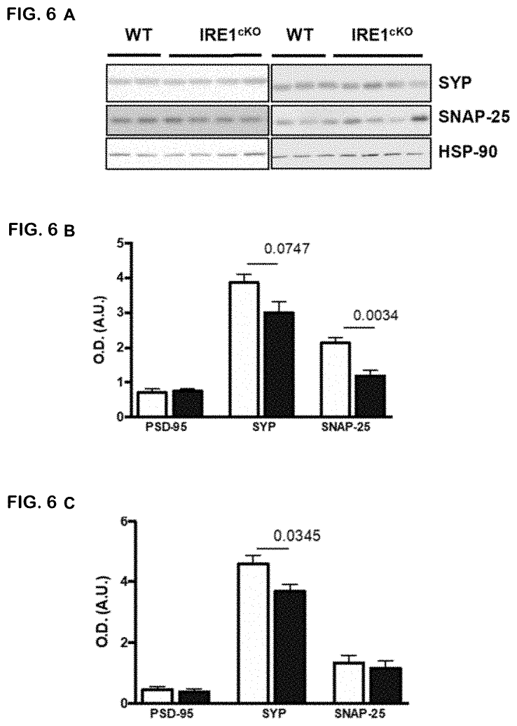

Interestingly, when comparing those strains during middle age, an early disruption of cognitive function in knock-outs was detected that was expected to happen only in advanced age, as evidenced in aged wild type animals in the new object recognition test (FIG. 5A). When analyzing old IRE1^{cKO} animals in the Barnes Maze test, we showed that they make more mistakes until they can find the target hole when compared to aged wild type mice (FIG. 5B). Such phenotype is absent during middle age or youth (FIG. 5B). Finally, we performed the Contextual Fear Conditioning (CFC) test in those strains to assess learning and memory with an alternative assay. Results indicate that although young IRE1^{cKO} present normal phenotype, during middle age those mice display significantly fewer freezing responses when compared to middle aged wild types (FIG. 5C). In conjunction, those findings indicate that ablating a functional IRE1-XBP1s pathway in the brain accelerates or even exacerbates age-associated cognitive disruption although it drives no further cognitive disability during youth. We also measured by western blot the levels of pre- and post-synaptic proteins in IRE1^{cKO} animals in different ages (FIGS. 6A, 6B, and 6C). Results indicate there is a significant decrease in the content of pre-synaptic proteins synaptophysin and SNAP-25 in the cerebral cortex and hippocampus of middle aged and old IRE1^{cKO} mice, which correlated with behavioral dysfunction in those time windows.

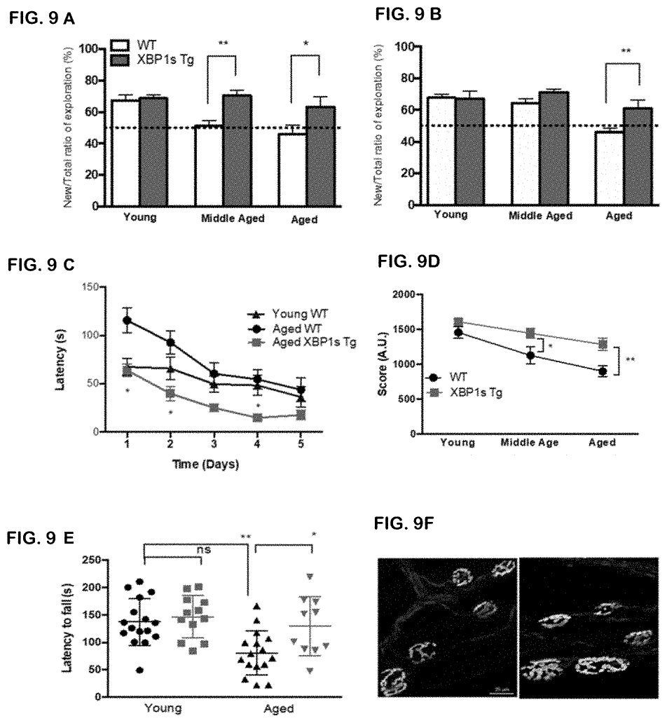

To assess the contribution of XBP1s to this process age matched conditional mutants overexpressing XBP1s in the brain, e.g. as described in WO2016/106458 and WO2016/106458, were evaluated using the same cognitive tests. Middle aged XBP1s transgenic mice present normal cognition when evaluated in the New Object Location (NOL) and New Object Recognition (NOR) tests, as expected for young wild type mice (FIGS. 9A and 9B). Remarkably, old XBP1s transgenic mice showed phenotype comparable to young wild types in the Barnes Maze as detected by the latency to find the target hole (FIG. 9C). These results indicate that XBP1s overexpression in the brain attenuates or even blocks the emergence of age-associated brain dysfunction.

### Example 3: IRE1-XBP1s Axis Modulates Age-Associated Motor Dysfunction

Experiments were also carried out using genetically modified mice for XBP1s (transgenic overexpressing active XBP1s in neurons) or IRE1 alpha (conditional knockout mice in the brain) and the normal cognitive and motor decline of mice during aging was evaluated. Remarkably, a functional role of brain UPR during aging is demonstrated here using classical gain- and loss-of-function experiments taking advantage of transgenic animals overexpressing XBP1s in the entire neuronal tissue and knock outs for the upstream UPR sensor IRE1 that could not express XBP1s in the brain. Following the aging course in those different mutant lines, it was observed that the IRE1-XBP1s pathway could alter age-associated cognitive and motor decline as shown by different tasks. Overall, the results indicate that therapeutic strategies to improve ER proteostasis are a possible candidate for interventions to reduce the risk of developing dementia and neurodegeneration and can also be used to ameliorate the normal motor and cognitive decay associated to the aging process.

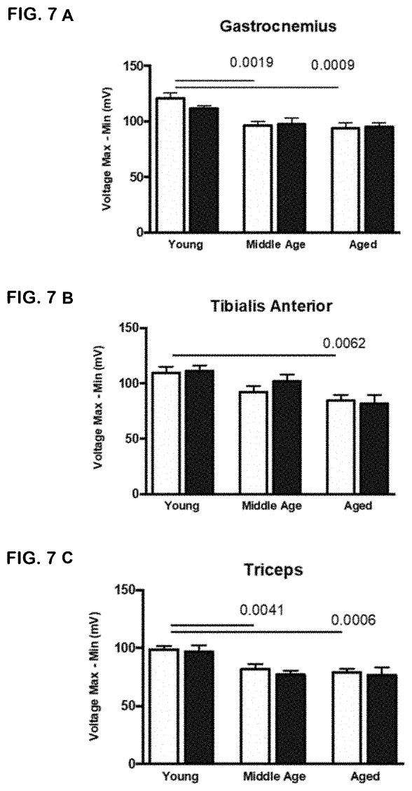

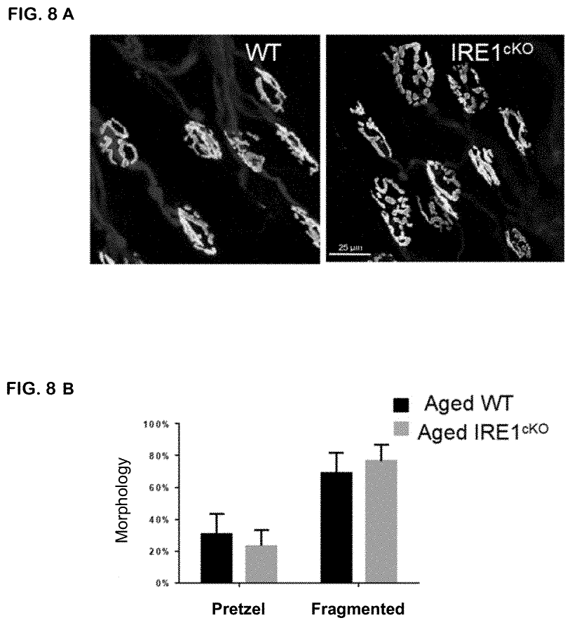

Comparing IRE1^{cKO} with wild type mice during youth, no significant differences were observed in motor function. However, old IRE1^{cKO} mice presented poorer motor function when compared to old wild types, as evidenced both in the rotarod test and the hanging test. These results suggest an age-dependent phenotype of IRE1 deficiency in motor dysfunction (FIGS. 5D and 5E). When evaluating the amplitudes of compound muscle action potentials (CMAPs) in three distinct muscle groups, aged wild type mice presented decreased amplitudes in all muscles analyzed (FIGS. 7A, 7B, and 7C). Of interest, there is no significant difference between wild types and IRE1^{cKO} animals in any age analyzed (FIGS. 7A, 7B, and 7C), indicating that age-dependent disruption observed in the knockout animals is likely dependent of higher motor circuits in the brain. Consistent with those findings, no significant alterations were observed in the morphology of neuromuscular junctions of mutant mice when compared to wild types (FIGS. 8A and 8B).

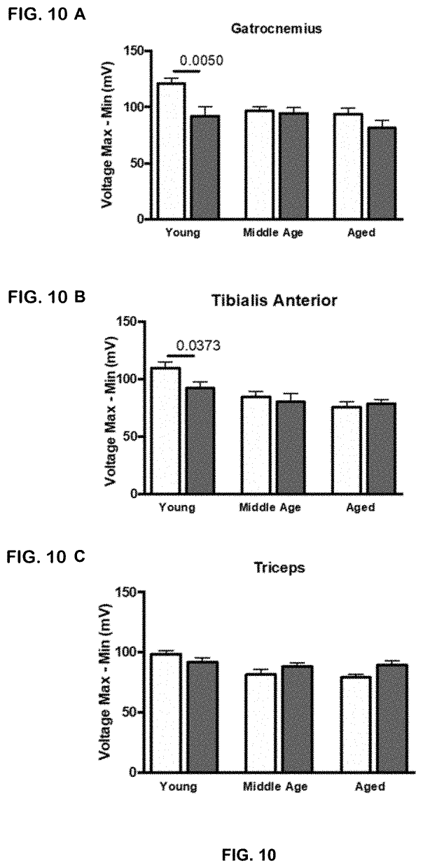

Age matched XBP1s transgenic mice were compared with wild-type animals to evaluate changes in motor disability during the course of aging. Remarkably, middle aged and aged transgenic animals presented better motor function when compared to wild types as shown by rotarod and hanging tests (FIGS. 9D and 9E) both in the hanging and rotarod tests although no changes could be measured during youth, thus suggesting that XBP1s overexpression in the brain could attenuate age associated motor dysfunction without any further alteration during youth. Again, no significant alterations were observed in the morphology of neuromuscular junctions in those mice (FIG. 9F). Young XBP1s transgenic mice presented slight decreased amplitudes in CMAP readings in the gastrocnemius and tibialis anterior muscles when compared to young wild types although no alterations were observed in middle aged or old animals (FIGS. 10A, 10B, and 10C).

These results indicate that the IRE1-XBP1s axis exerts a major role in sustaining brain health span during aging in mammals. Thus, targeting this pathway may prove a powerful tool to modulate undesired outcomes of the aging process thus increasing health and life quality of the elderly population.

### REFERENCES

1. Houtkooper R H, Argmann C, Houten S M, Cant6 C, Jeninga E H, Andreux P A, et al. The metabolic footprint of aging in mice. Sci Rep (2011) 1.
2. Petersen R C. Mild cognitive impairment as a diagnostic entity. Journal of Internal Medicine (2004) 256(3):183-94.
3. Brayne C. The elephant in the room—healthy brains in later life, epidemiology and public health. Nat Rev Neurosci (2007) Mar. 8(3):233-9.
4. Sanford, Sarah, et al. Independence, loss, and social identity: Perspectives on driving cessation and dementia. Dementia (2018):1471301218762838.
5. Kaushik S, Cuervo AM. Proteostasis and aging. Nature Medicine (2015) 21(12):1406-15.
6. Martinez, Gabriela et al. Endoplasmic reticulum proteostasis impairment in aging. Aging Cell (2017) Aug.; 16(4): 615-623.
7. Hetz C. The unfolded protein response: Controlling cell fate decisions under ER stress and beyond. Nat Rev Mol Cell Biol (2012) 13(2):89-102.
8. Hetz, C., & Saxena, S. ER stress and the unfolded protein response in neurodegeneration. Nature reviews. Neurology (2017) Aug. 13(8):477-491.
9. Valdes P, Mercado G, Vidal R L, Molina C, Parsons G, Court F A, et al. Control of dopaminergic neuron survival by the unfolded protein response transcription factor XBP1. Proc Natl Acad Sci USA (2014) May 6;111(18):6804-9.
10. Hetz C, Thielen P, Matus S, Nassif M, Court F, Kiffin R, et al. XBP-1 deficiency in the nervous system protects against amyotrophic lateral sclerosis by increasing autophagy. Genes Dev (2009) Oct. 1;23(19):2294-306.
11. Vidal R L, Figueroa A, Court F A, Thielen P, Molina C, Wirth C, et al. Targeting the UPR transcription factor XBP1 protects against huntington's disease through the regulation of foxol and autophagy. Hum Mol Genet (2012) May 15;21(10):2245-62.
12. Duran-Aniotz, C., Cornejo, V. H., Espinoza, S., Ardiles, A. O., Medinas, D. B., Salazar, C., & Scheper, W. IRE1 signaling exacerbates Alzheimer's disease pathogenesis. Acta Neuropathologica (2017) Sep.; 134(3):489-506.
13. LIU, Sheng-Yuan, et al. Polymorphism-116 C/G of Human X-box-Binding Protein 1 Promoter is Associated with Risk of Alzheimer's Disease. CNS neuroscience & therapeutics (2013) 19(4):229-234.
14. Martinez, Gabriela, et al. “Regulation of memory formation by the transcription factor XBP1.” Cell reports (2016) 14(6):1382-1394.
15. Rampon, C., et al. Effects of environmental enrichment on gene expression in the brain. Proc. Natl. Acad. Sci. USA (2000) 97:12880-12884.
16. Hayashi, A., et al. The role of brain-derived neurotrophic factor (BDNF)-induced XBP1 splicing during brain development. J. Biol. Chem. (2007) 282:34525-34534.
17. Hayashi, A., Kasahara, T., Kametani, M., and Kato, T. Attenuated BDNF-induced upregulation of GABAergic markers in neurons lacking Xbp1. Biochem. Biophys. Res. Commun. (2008) 376:758-763.
18. Taylor R C, Dillin A. XBP-1 is a cell-nonautonomous regulator of stress resistance and longevity. Cell (2013) 153(7):1435-47.
19. LUIS, Nuno Miguel, et al. Intestinal IRE1 is required for increased triglyceride metabolism and longer lifespan under dietary restriction. Cell reports (2016)

1. A method for the delay or treatment of a symptomatic stage of aging in a subject, the method comprising administering to the subject a therapeutically effective amount of a pharmaceutically acceptable composition comprising X-box binding protein 1 (XBP1) or an agent that stimulates expression of a polypeptide comprising XBP1 in the brain of the subject to maintain or restore endoplasmic reticulum proteostasis in the subject.

2. The method of claim 1, wherein the composition comprises an adeno-associated virus (AAV) vector.

3. The method of claim 2, wherein the AAV vector induces expression of XBP1s.

4. The method of claim 2, wherein the AAV vector induces expression of a fusion protein comprising XBP1 and ATF6, optionally joined by a linker.

5. The method of claim 1, wherein the XBP1 is a mammalian XBP1, preferably human XBP1.

6. The method of claim 4, wherein the ATF6 is a mammalian ATF6, preferably human ATF6.

7. The method of claim 2, wherein the AAV vector is of a serotype selected from the group consisting of AAV2, AAV6, AAV7, AAV8 and AAV9.

8. The method of claim 7, wherein the AAV vector is of the serotype AAV2 or AAV6.

9. The method of claim 1, wherein the symptomatic stage of aging is a decline in basal motor and/or cognitive function associated with aging.

10. The method of claim 1, wherein administration of the composition substantially prevents, reduces, reverses or delays decay in basal cognitive and/or motor capacity.

11. The method of claim 1, wherein the composition is administered to an aged mammalian subject.

12. The method of claim 11, wherein the subject is suffering from age-related cognitive decline.

13. The method of claim 1, wherein the subject is human.

14. The method of claim 1, wherein the pharmaceutically acceptable composition is administered systemically or locally.

15. The method of claim 1, wherein the pharmaceutically acceptable composition is administered by a nasal route or by direct intraventricular or intrathecal injection and the composition passes the haemato-encephalic barrier.

16. The method of claim 1, wherein the agent stimulates expression of a polypeptide comprising XBP1 in the hippocampus.

17. A method for the prevention or treatment of an age-related disorder in a subject, the method comprising administering to the subject a therapeutically effective amount of X-box binding protein 1 (XBP1) or an agent that stimulates or induces expression or over-expression of XBP1 in the brain.

18. The method of claim 17, wherein the disorder is age-related cognitive decline.

19. The method of claim 17, wherein the disorder is age-related motor dysfunction.

20. The method of claim 17, wherein the disorder is progeria or accelerated ageing.

21. The method of claim 19, wherein the agent comprises an adeno-associated virus (AAV) vector.

22. The method of claim 21, wherein the AAV vector induces expression of XBP1s.

23. The method of claim 21, wherein the AAV vector induces expression of a fusion protein comprising XBP1 and ATF6, optionally joined by a linker.

24. The method of claim 17, wherein the XBP1 is a mammalian XBP1, preferably human XBP1.

25. The method of claim 23, wherein the ATF6 is a mammalian ATF6, preferably human ATF6.

26. The method of claim 21, wherein the AAV vector is of a serotype selected from the group consisting of AAV2, AAV6, AAV7, AAV8 and AAV9.

27. The method of claim 26, wherein the AAV vector is of the serotype AAV2 or AAV6.

28. The method of claim 17, wherein the agent is administered to a mammalian subject suffering from an age-related disorder.

29. The method of claim 28, wherein the subject is human.

30. The method of claim 17, wherein a pharmaceutically acceptable composition comprising XBP1 or the agent is administered systemically or locally to the subject.

31. The method of claim 30, wherein the pharmaceutically acceptable composition is administered by a nasal route or by direct intraventricular or intrathecal injection and the composition passes the haemato-encephalic barrier.

32. The method of claim 17, wherein the agent stimulates expression of a polypeptide comprising XBP1 in the hippocampus.

33. The method of claim 1, wherein the polypeptide comprises an amino acid sequence having at least 85%, at least 90%, at least 99% or at least 99% sequence identity to SEQ ID NO:1 or SEQ ID NO:2.

34. The method of claim 17, wherein the polypeptide comprises an amino acid sequence having at least 85%, at least 90%, at least 99% or at least 99% sequence identity to SEQ ID NO:1 or SEQ ID NO:2.
